(19) United States
(12) Patent Application Publication
(10) Pub. No.: US 2003/0206973 A1
(43) Pub. Date: Nov. 6, 2003

(54) All natural gentle deodorant and antiperspirant

(76) Inventor: Carolyn Gale, Chicago, IL (US)

Correspondence Address: JoAnne M. Denison, Suite 1608, 212 W. Washington Blvd., Chicago, IL, 60606-3487, US

(21) Appl. No.: 10/138,852

(22) Filed: May 1, 2002

(51) Int. Cl. A61K 35/78; A61K 7/32

(52) U.S. Cl. 424/725; 424/65

(57) ABSTRACT

The present invention consists of and all natural deodorant/antiperspirant which contains olive oil, almond oil, wheat germ oil as gentle soothing emolients, salvia officianalis and cupressus semperiens as natural astringents, lavendula angustifolio and citrus aurantium as natural, mild antiseptic compounds. Beeswax or hydrogenated castor oil may be added as a gentle thickening agent where the inventive formulation is to be used in cream or solid form. In such a manner, skin irritation, swelling, occlusions and inflamations are prevented.

| | Preferred | Permissible Range |
|---|---|---|
| Ingredient | Embodiment | (volume % given) |
| 100% Olive Oil | 29.2% | 27.2-31.2% |
| 100% Almond Oil | 43.8% | 41.8-45.8% |
| 100% Wheat Germ Oil | 21.9% | 19.9-23.9% |
| Beeswax | 2.9% | 2.7-3.1% |
| Salvia Officinalis | 0.73% | 0.71-0.75% |
| Lavendula Angustifolio | 0.58% | 0.56-0.60% |
| Citrus Aurantium | 0.44% | 0.42-0.46% |
| Cupressus Semperiens | 0.44% | 0.42-0.46% |

### FIELD OF THE INVENTION

The present invention relates to the field of deodorants and antiperspirants, and more particularly those which are all natural in composition and therefore gentle and non-irritating to most skin types and which have a gentle, pleasing mild odor.

### BACKGROUND OF THE INVENTION

Currently, in the marketplace, there are available a wide variety of deodorants/antiperspirants which are intended to mask natural underarm orders, however most of these deodorants have unnatural substances or man made chemicals or fragrances, which are irritating to the user. Very young or very old persons often have delicate skin and pores are especially susceptible to skin rashes, sores and pores clogging due to all of the unnatural chemicals and fragrances found in commercially available deodorants.

As a good example of a typical commercially available deordorant/atniperspiratnt, Secret brand deodorant contains 19% Aluminum Zirconium Trichlorohydrox as its active ingredient and Cycloropentasiloxane dimethicon, Tribehenin C₁₈₋₃₆ and Triglycerine, and “fragerance” as other ingredients. All of these ingredients are not natural and may cause skin irritation and breakdown of the skin. Similarly Lady Mitchum deodorant contains 17% Aluminum Zirconium Tetrachlorohyrox, dipolypropylene glycol, hydrosteric acid, dimenticone copolyol, cycomethicone, agarose, acetamide MEA, dimethicone cross polymer and fragrance, all of which may cause skin irritation or skin breakdown with some users. Suave brand-deodorant contains cyclopentasiloxane, PPG-14, butyl ether, steryl alcohol, hydrogenated castor oil, PEG-8, disterate, talc, fragrance, vegetable oil, glycerol oleate, PPG, BHT, t-butyl hydroquinone, citric acid, most or almost all of which are known skin irritants or are substances known to cause skin breakdown in sensitive individuals.

Some commercially available deodorants/antiperspirants claim that they are “nautural” or contain natural ingredients. But together with commonly known natural ingredients, such as aloe or tea tree oil, they also contain a plethora of unnaturally occurring chemicals, fragrances and additives. Almay brand deodorant, which is a manufacturer known to make hypoallergenic claims regarding its beauty products, lists ingredients similar to other commercial deodorant brands: 17% aluminum zirconium tetrachloro hydrox, di-polypropylene glycol, cyclomethicone, dimethicone, glycerine, dimethicone copolyol, all of which are known to cause skin irritations and breakdown in some sensitive individuals.

Suave brand “Naturals” contains aluminum zirconium tetra GLY, stearyl alcohol, hydrogenated castor oil, PEG-8 disterate, fragerance, tocopheryl acetate, vitamin E acetate, sodium bicarbonate, vegetable oil, glyceral oleate, PPG t-butyl hydroquinone and citric acid and many of these chemicals are known skin irritants or known to break down the skin in some sensitive individuals.

One brand of a more natural deodorant in the marketplace is Tom's of Maine which lists much more natural ingredients such as camomile tea, aloe vera, lichen extract, witch hazel and coriander oil, but it also lists unnatural chemicals such as polypropylene glycol, sodium sterate and glyceryl monolaurate., all of which are known to be skin irritants or which may break down the skin in certain sensitive individuals.

Another product which is claimed to be natural is a Tea Tree Oil Deodorant Jel which is made by Earthwise which contains vegetable oil (glycerine), extract of hamameleris, grain alcohol, aloe, grapefruit seed extract, tea tree oil, extract of arnica, camomile and calendula. Yet another product by Queen Helene, a Vitamin E deodorant, contains polyprolyene glycol, water, sodium sterate, tocopheryl acetate, triclosan and natural fragrances. Jason brand makes an Apricot Oil deodorant which is claimed to be “natural” which contains polypropylene glycol, sodium stearate, soyaethyl morpholinian, ehto sulfate, allantoin, tocoperol, baking soda, purified clay, honey suckle, apricot, peach and passionfruit extracts as fragrances, panthehol (B-5), citric acid and methyl paraben, a preservative. Jason also makes a brand of tea tree oil roll on deodorant which contains glycerin (vegetable), vetetabke starches, octyl palmitate, purified clary, glyceral sterate, allantoin, steryl alcohol, zinc riconieate, dl-pantheonol, xanathan gum, tocoperol, soy aethyl morpholinium, ethosulfate, dicaprylate-dicapriate, potassium sorbate, tea tree oil, natural fragrances—kiwi, lilac, hyacinth, rose, spice, lavender and seaweek, methylparaben and porpyl paraben.

Laviun makes a “long lasting” deodorant which contains zinc oxide, petroleum jelly, talc, potato starch, calendula oil, arnica oil, vitamin E, ascorbyl palmitate, citric acid and natural fragrances. Likewise, NOW brand solid deodorant contains zinc oxide, propylene glycol, sodium sterate, grapefruit extract, marigold extract, arnica extract and lavender oil. Desert Essence makes a solid deodorant containing polypropylene glycol, purified water, sodium sterate, cocomide DEA, aloe barbadenis gel, neem oil, camomile extract, matricaria extract, calendula extract, witch hazel extract, lichen extract and sodium borate.

Another deodorant described as “all natural” is made by Dessert Essence and is called “Tea Tree Oil and Sage” deodorant and it a roll on containing purified water, glyceryl stearate, polysorbate 20, vegetable emulsifying wax, tea tree oil, sodium bicarbonate, methyl paraben, famesol, sage, thyme, burdock, oak bark, lemon extract, aloe vera, camomile extract, calendula extract and lemongrass oil.

Various patents in the prior art discuss methods by which natural ingredients may be used to produce an all natural deodorant/antiperspirant. In U.S. Pat. No. 6,139,825 issued to Reinhard, et al., it is discussed that sage, or Salvia Officinalis, has leaves and flowers which may produce an effective deodorant. It is further known from U.S. Pat. No. 4,942,033 that a mixture of extracts from Butcher's broom or Ruscus aculeatur L. and sage is effective in reducing capillary permeability and thus has an anti-hidrotic effect and will accordingly reduce production of sweat when applied to the skin. U.S. Pat. No. 6,139,825 describes the use of a particular CO₂ process to more effectively extract compounds necessary to produce a sage type deodorant from the plant's leaves and flowers. However, this patent advises the use of conventional carriers and substances to produce the final form of the deodorant/antiperspirant, thus producing a product with chemicals and substances which may irritate the skin of sensitive users of the product.

In U.S. Pat. No. 6,001,341 is described the different substances commonly used in deodorants/antiperspirants and how each of the particular chemical substances may cause skin irritation and breakdown: 1) bactericidal substances can cause allergies, skin breakdown and may interfere with the cellular membrane structures of skin cells; 2) perfumes which merely mask odor may interfere by occluding armpit pores and entering the body through the armpit pores resulting in irritation and inflamation; 3) odor-absorbing substances may produce occlusions; 4) volatile alcohols may produce skin irritation, dehydration of the skin and allergic reactions including rash and inflammation; and 5) astringent substances may produce skin irritation, dehydration of the skin and allergic reactions including rash and inflammation.

U.S. Pat. No. 5,976,514 issued to Guskey et al. discloses a formulation for an antiperspirant/deodorant which utilizes popular nonpolar hydrocarbonic liquids, which while often producing skin irritation, are combined with selected mitigating materials preferably consisting of a nonvolatile, nonpolar hydrocarbon emollient and/or a nonvolatile silicone material, preferably nonvolatile silicone fluid, in select weight ration and vapor pressures in order to greatly reduce the instance of skin irritation and inflamation and occlusion in the user. However, this particular disclosure relies on the utilization of standard chemicals and substance in a deodorant/antiperspirant formulation which are merely mixed with certain unnatural, chemicals and substances which are shown to mitigate undesired effects of skin irritation, inflammation and skin break down.

U.S. Pat. No. 5,814,310 teaches the use of jojoba oil and a soap gelling agent as the carrying agent for a solid cosmetic stick to reduce the incidence of adverse skin reactions when chemicals and other unnatural substances are applied to the skin, but it does not teach an all natural method or composition suitable for use as a deodorant or antiperspirant.

In U.S. Pat. No. 4,933,177 is taught the method and formulation of cosmetic compositions for the treatment of hair and skin which contain 5 to 30 percent by weight of pulverized plant materials having astringent properties selected from a group consisting of acacia, witch hazel, tormentil, dog rose and rhatany. In examples 33 and 34 are given formulations of antiperspirants and deodorants which utilize natural ingredients of rosemary leaves and chestnut tree powder, but each of these formulations utilize a number of standard, unnatural chemicals and substances used in typical commercial deodorant and antiperspirant formulations.

Hence, most or almost all of the above patents do not teach of a deodorant made from all natural substances, and in particular a deodorant/antiperspirant which is not only comprised of all natural ingredients, but those ingredients chosen naturally inhibit the growth of bacteria, which prevents odor, they are highly aromatic without being especially particularly allergenic, and they are further capable of producing an anti-hydrotic effect by naturally restricting the flow of water or moisture out the skin pores of the user.

### SUMMARY OF THE INVENTION

The present invention consists of a mild, gentle deodorant and antiperspirant which is composed of all natural ingredients and is free of unnatural chemicals and substances which typically produce skin swelling, irritation, occlusion of pores and inflammation. The present invention contains no volatile silicones or nonpolar hydrocarbonic liquids which are well known in the prior art to produce the skin problems previously mentioned. The ingredients utilized in this preferred embodiments of the present invention were carefully selected for desirable properties inducive to preventing skin irritation, inflammation, pore occlusion and swelling and are all natural. The ingredients chosen actually promote and enhance skin health. They produce odor control by reducing sweat action through their natural astringency, or pore closing properties, as well as their antiseptic properties by reducing unwanted bacterial growth which produces odor. At the same time, they limit their action on the beneficial or “probacterias” found naturally in healthy skin, so as to minimize or prevent any skin breakdown by the user.

Although the ingredients used in this particular embodiment are to be used in a roll on container, other methods commonly known in the industry may be employed to produce the antiperspirant/deodorant in cream, solid or gel form. When the solid or cream form is chosen, it is anticipated that the carrying agent also be as all natural as possible, such as hydrogenated castor oil or beeswax, or other such similar agent which is solid in form at room temperature.

Accordingly, the preferred embodiment for the present invention is as follows:

| | Preferred | Permissible Range |
|---|---|---|
| Ingredient | Embodiment | (volume % given) |
| 100% Olive Oil | 29.2% | 27.2-31.2% |
| 100% Almond Oil | 43.8% | 41.8-45.8% |
| 100% Wheat Germ Oil | 21.9% | 19.9-23.90% |
| Beeswax | 2.9% | 2.7-3.1% |
| Salvia Officinalis | 0.73% | 0.71-0.75% |
| Lavendula Angustifolio | 0.58% | 0.56-0.60% |
| Citrus Aurantium | 0.44% | 0.42-0.46% |
| Cupressus Semperiens | 0.44% | 0.42-0.46% |

With respect to the above ingredients, each performs an important role in producing an optimal deodorant/antiperspirant which is gentle and promotes health skin. Olive oil has been chosen because it is high in essential fatty acid content in order to produce a soothing, lubricating effect on the skin. It is also preferred as an ingredient for its stability and nutrient value to the skin in that it contains high levels of antioxidants such as beta carotene and vitamin E.

Almond Oil is also a preferred ingredient for an all natural formulation for an deodorant and/or antiperspirant inasmuch as it rich in Vitamin E and is a well known favored treatment for dry, itching, inflamed skin. Likewise, wheat germ oil is a preferred ingredient for the present invention in that it contains essential fatty acids, beta carotene and vitamin E. Salvia Officianalis has been choosen as a preferred ingredient for the present invention in that it comprises a natural mild astringent that mimics performance of an antiperspirant without unnatural or foreign chemicals or substances. Similarly Lavendula Angustifolio is highly antiseptic, yet gentle and is known to promote and facilitate healthy skin. Citrus Aurantium is also known for its properties of bringing balance and strength to damaged, dry skin. Similarly, Cupressus Semperiens is known for its natural yet mild astringent qualities which inhibit the growth of odor producing bacteria.

### OBJECTS OF THE INVENTION

Thus, it is one primary object of the present invention to provide a most gentle and pleasing deodorant/antiperspirant which consists of all natural ingredients including the use of olive oil which is high in essential fatty acids for a soothing, lubricating effect on the skin.

It is yet another primary object of the present invention to provide a most gentle and pleasing deodorant/antiperspirant which consists of all natural ingredients including the use of olive oil for its nutrient properties in that it naturally contains antioxidants, beta carotene and vitamin E.

It is yet a further primary object of the present invention to provide a most gentle and pleasing deodorant/antiperspirant which consists of all natural ingredients including the use of Almond oil which contains much vitamin E and is known for healing properties for dry, itchy and/or inflamed skin.

Still an additional primary object of the present invention to provide a most gentle and pleasing deodorant/antiperspirant which consists of all natural ingredients including the use of wheat germ oil which contains fatty acids for hydration of the skin, as well as nutrients beta carotene and vitamin E to promote healthy, strong skin.

Still a further primary object of the present invention to provide a most gentle and pleasing deodorant/antiperspirant which consists of all natural ingredients including the use of Salvia Officianalis which is a natural mild astringent that reduces the incidence of sweat by constricting skin pores hence reducing malodors produced by extraneous bacteria on the skin surface.

Yet an additional primary object of the present invention to provide a most gentle and pleasing deodorant/antiperspirant which consists of all natural ingredients including the use of Lavendula Angustifolio which is a natural antiseptic which inhibits the growth of extraneous bacteria on the skin surface and it further induces maintenance of strong, healthy skin.

Still another primary object of the present invention to provide a most gentle and pleasing deodorant/antiperspirant which consists of all natural ingredients including the use of Citrus Aurantium which naturally balances the skin chemistry and induces maintenance of strong, healthy skin.

And yet antoher primary object of the present invention to provide a most gentle and pleasing deodorant/antiperspirant which consists of all natural ingredients including the use of Cupressus Semperiens which also is a natural astringent that reduces or reduces the incidence of sweat and hence extraneous bacterial growth.

These and other objects and advantages of the present invention can be readily derived from the following detailed description of the instant invention present herein and should be considered as within the overall scope of the invention.

### DETAILED DESCRIPTION OF THE PRESENT INVENTION

As described above, it is anticipated that the following ingredients are used in a preferred embodiment of the present invention:

| | Preferred | Permissible Range |
|---|---|---|
| Ingredient | Embodiment | (volume % given) |
| 100% Olive Oil | 29.2% | 27.2-31.2% |
| 100% Almond Oil | 43.8% | 41.8-45.8% |
| 100% Wheat Germ Oil | 21.9% | 19.9-23.9% |
| Beeswax | 2.9% | 2.7-3.1% |
| Salvia Officinalis | 0.73% | 0.71-0.75% |
| Lavendula Angustifolio | 0.58% | 0.56-0.60% |
| Citrus Aurantium | 0.44% | 0.42-0.46% |
| Cupressus Semperiens | 0.44% | 0.42-0.46% |

The olive oil was chosen for its high essential fatty acid content as well as the presence of a great amount of antioxidants and beta carotene and vitamin E. The almond oil was selected for its high content of Vitamin E which promotes healthy skin, as well as its ability to soothe dry, itchy or inflamed skin. The wheat germ oil had been selected for the presence of essential fatty acids, beta carotene and vitamin E content, all of which promote moist, healthy, strong skin. Salvia Officianalis was selected for its very mild, natural astringent properties which naturally inhibit or reduce the incidence of sweat by closing the skin pores. Lavendula Angustifolio was selected for its highly antiseptic properties which inhibit the growth of malodorous bacteria on the skin surface. Citrus Aurantium was selected for its ability to balance and tone the skin and promote skin strength. Finally, Cupressus Semperiens was selected for its natual, mild astringent quality which inhibits the production of sweat by closing up skin pores.

It is anticipated that the above mild deodorant/antiperspirant composition may be applied topically to the axilla or other areas of the skin for malodor control The present invention is preferably applied in an amount ranging from about 0.1 to 10 grams to the desired areas of the skin, once or twice daily or as needed. The Salvia Officinalis, Lavendula Angustifolio, Citrus Aurantium and Cupressus Semperiens may be comprised of finely chopped or crushed dried plant materials, or they may consist of extracts from the plants produced through drying or crushing processes. All of the ingredients are mixed or blended together to produce the proper consistency. Where a roll on is desired, the mixture can be thinned, if desired with pure or filtered water, so as not to produce skin irritation. When a stick or solid form of the antiperspirant/deodorant is desired, additional beeswax or hydrogenated castor oil or a similar all natural carrying agent may be added so that the formulation is solid at room temperature. Both of these methods are well known in the prior art.

Although in the foregoing detailed description the present invention has been described by reference to various specific embodiments, it is to be understood that modifications and alterations in the structure and arrangement of those embodiments other than those specifically set forth herein may be achieved by those skilled in the art and that such modifications and alterations are to be considered as within the overall scope of this invention.

What is claimed is:

1. A natural composition containing at least one natural oil selected from the following group: olive oil, almond oil or wheat germ oil;
at least one natural astringent selected from the following group: Salvia Officinalis or Cupressus Semperiens; and,
at least one natural antiseptic agent selected from the following group: Lavendula Angustifolio or Cupressus Semperiens.

2. The natural composition according to claim 1 further having a thickening agent.

3. The natural composition according to claim 2 wherein the thickening agent is selected from the following group: beeswax or hydrogenated castor oil.

4. The natural composition according to claim 1 which contains both Salvia Officinalis and Cupressus Semperiens.

5. The natural composition according to claim 1 which contains both Lavendula Angustifolio and Cupressus Semperiens.

6. The natural composition according to claim 5 which contains Salvia Officinalis, Cupressus Semperiens, Lavendula Angustifolio and Curpessus Semperiens.

7. The natural composition according to claim 6 which contains Salvia Officinalis in the volume % amount from 27.2-32.2%, Cupressus Semperiens in the volume % amount from 0.42-0.46%, Lavendula Angustifolio in the volume % amount of 0.56-0.60% and Citrus Aurantium in the volume % amount of 0.42-0.46%.

8. The natural composition according to claim 6 which Salvia Officinalis in the volume % amount substantially from 20-40%, Cupressus Semperiens in the volume % amount from 0.05-2.5%, Lavendula Angustifolio in the volume % amount of 0.05-5.00% and Citrus Aurantium in the volume % amount of 0.05-5.0%.

9. The natural composition according to claim 1 in which is present each of the olive oil, almond oil and wheat germ oil.

10. The natural composition according to claim 1 in which is present each of: olive oil, almond oil, wheat germ oil, salvia officianalis, lavendula angustifolio, citrus aurantium, and cupressus semperiens.
